(19) United States
(12) Patent Application Publication
(10) Pub. No.: US 2023/0335277 A1
(43) Pub. Date: Oct. 19, 2023

(54) ARTIFICIAL INTELLIGENCE BASED SYSTEM AND METHOD FOR AUTOMATICALLY MONITORING THE HEALTH OF ONE OR MORE USERS

(71) Applicant: Neural Foundry Limited, Slough (GB)

(72) Inventor: Venkata Yagna Dhruva Kumar Konidena, Slough (GB)

(21) Appl. No.: 17/720,536

(22) Filed: Apr. 14, 2022

(51) Int. Cl. G16H 50/20; A61B 5/11; G06V 20/40; G06V 20/70; G06T 1/00; G10L 17/22; G16H 80/00

(52) U.S. Cl. CPC G16H 50/20; A61B 5/1128; G06V 20/44; G06V 20/70; A61B 5/1117; A61B 5/1113; G06T 1/0014; G10L 17/22; G16H 80/00

(57) ABSTRACT

An AI-based system and method for automatically monitoring the health of one or more users is disclosed. The method includes capturing one or more videos of one or more users and extracting a plurality of frames from each of the one or more videos. The method includes extracting a set of skeletal positions from each of the plurality of frames and performing one or more operations on the plurality of frames to normalize the set of skeletal positions. Furthermore, the method includes detecting a set of poses of the one or more users in the plurality of frames and determining an action performed by the one or more users in the plurality of frames by using an action determination-based AI model. The method includes determining a level of severity of the action and performing one or more responsive actions to provide medical assistance to the one or more users.

### FIELD OF INVENTION

Embodiments of the present disclosure relate to healthcare robotic systems and more particularly relates to an AI-based system and method for monitoring the health of one or more users.

### BACKGROUND

Generally, most elderly people i.e., aged 65 or above, live alone or with their spouse. Thus, it is very important to monitor daily activities of elderly people to detect emergencies, such as a bad fall, a medicine overdose, and the like, and then report the detected emergency to emergency contacts, such as a health care professional or first responders. Often, elderly people live in nursing homes or appoint healthcare professionals to receive help related to personal care. However, the conventional solutions of living in nursing homes and appointing the healthcare professionals for receiving help related to the personal care are very expensive. Further, loneliness is a common condition for certain classes of people, such as elderly people, disabled people, and the like. Specifically, elderly people are at a high risk of loneliness and social isolation, as most of them are living alone. There are various medical consequences of loneliness, such as altered brain function, Alzheimer's disease progression, antisocial behavior, depression, and the like. Thus, it is important to timely treat the loneliness condition for betterment of these classes of people.

Hence, there is a need for an improved AI-based system and method for automatically monitoring the health of one or more users in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, an AI-based computing system for automatically monitoring the health of one or more users is disclosed. The AI-based computing system includes one or more hardware processors and a memory coupled to the one or more hardware processors. The memory includes a plurality of modules in the form of programmable instructions executable by the one or more hardware processors. The plurality of modules include a video capturing module configured to capture one or more videos of one or more users by using one or more imaging capturing devices. A plurality of frames are extracted from each of the captured one or more videos by using a frame extraction technique. The plurality of modules also include a position extraction module configured to extract a set of skeletal positions from each of the extracted plurality of frames by using a position extraction-based Artificial Intelligence (AI) model. The plurality of modules includes an operation performing module configured to perform one or more operations on the extracted plurality of frames based on the extracted set of skeletal positions and one or more image parameters to normalize the extracted set of skeletal positions. Further, the plurality of modules includes a pose detection module configured to detect a set of poses of the one or more users in the plurality of frames based on the normalized set of skeletal positions and predefined pose information by using an action determination-based AI model. The plurality of modules also include an action determination module configured to determine an action from one or more actions performed by the one or more users in the extracted plurality of frames based on the normalized set of skeletal positions, the detected set of poses and the predefined pose information by using the action determination-based AI model. The one or more actions include: sitting, standing, slipping, tripping and falling. Furthermore, the plurality of modules include a severity level determination module configured to determine a level of severity of the determined action based on reaction of the one or more users by using an offline voice management-based AI model. The plurality of modules include an action performing module configured to perform one or more responsive actions if the determined level of severity is above a predefined threshold level based on the determined action and the determined level of severity to provide medical assistance to the one or more users.

In accordance with another embodiment of the present disclosure, an AI-based method for automatically monitoring the health of one or more users is disclosed. The AI-based method includes capturing one or more videos of one or more users by using one or more image capturing devices. A plurality of frames are extracted from each of the captured one or more videos by using a frame extraction technique. The AI-based method further includes extracting a set of skeletal positions from each of the extracted plurality of frames by using a position extraction-based Artificial Intelligence (AI) model. Further, the AI-based method includes performing one or more operations on the extracted plurality of frames based on the extracted set of skeletal positions and one or more image parameters to normalize the extracted set of skeletal positions. Also, the AI-based method includes detecting a set of poses of the one or more users in the plurality of frames based on the normalized set of skeletal positions and predefined pose information by using an action determination-based AI model. Furthermore, the AI-based method includes determining an action from one or more actions performed by the one or more users in the extracted plurality of frames based on the normalized set of skeletal positions, the detected set of poses and the predefined pose information by using the action determination-based AI model. The one or more actions include: sitting, standing, slipping, tripping and falling. The AI-based method also includes determining a level of severity of the determined action based on reaction of the one or more users by using an offline voice management-based AI model. Further, the AI-based method includes performing one or more responsive actions if the determined level of severity is above a predefined threshold level based on the determined action and the determined level of severity to provide medical assistance to the one or more users.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for automatically monitoring the health of one or more users, in accordance with an embodiment of the present disclosure;

FIG. 2 is a block diagram illustrating an exemplary AI-based computing system for automatically monitoring the health of the one or more users, in accordance with an embodiment of the present disclosure;

FIG. 3A is a top or sectional view of the exemplary AI-based computing system, in accordance with an embodiment of the present disclosure;

FIG. 3B is a front view of the exemplary AI-based computing system, in accordance with an embodiment of the present disclosure;

FIG. 3C is a side view of the exemplary AI-based computing system, in accordance with an embodiment of the present disclosure;

FIG. 3D is an isometric view of the exemplary AI-based computing system, in accordance with an embodiment of the present disclosure;

FIG. 3E is a detailed view of exemplary ergonomically placed grips, in accordance with an embodiment of the present disclosure;

FIG. 4 is an exemplary block diagram depicting operation of the AI-based computing system to determine an action from one or more actions, in accordance with an embodiment of the present disclosure;

FIG. 5 is an exemplary block diagram depicting operation of the AI-based computing system to perform one or more responsive actions and output one or more speech outputs, in accordance with an embodiment of the present disclosure;

FIG. 6 is a process flow diagram illustrating an exemplary AI-based method for automatically monitoring the health of the one or more users, in accordance with an embodiment of the present disclosure; and

FIGS. 7A-7D are graphical user interface screens of the AI-based computing system for automatically monitoring the health of the one or more users, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

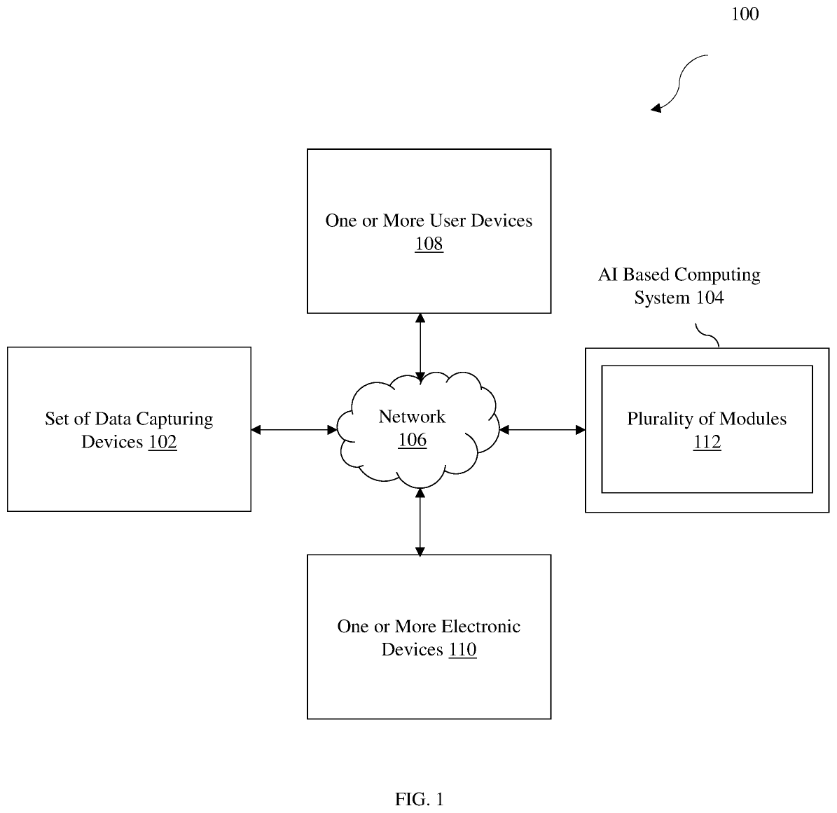

Referring now to the drawings, and more particularly to FIGS. 1 through FIG. 7D, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 for automatically monitoring the health of one or more users, in accordance with an embodiment of the present disclosure. According to FIG. 1, the computing environment 100 includes a set of data capturing devices 102 communicatively coupled to an AI-based computing system 104. In an embodiment of the present disclosure, the set of data capturing devices 102 are placed inside the AI-based computing system 104. In another embodiment of the present disclosure, the set of data capturing devices 102 are placed in proximity of one or more users. The set of data capturing devices 102 may be Internet of Things (IOT) sensors. For example, the set of data capturing devices 102 are fixed on interior house walls of the one or more users to capture data associated with the one or more users. The one or more users may include any class of people, such as elderly people, teenagers, disabled people and the like. In an exemplary embodiment of the present disclosure, the data associated with the one or more users include one or more videos, one or more audio inputs, one or more images, motion of the one or more users and the like. In an embodiment of the present disclosure, the set of data capturing devices 102 are communicatively coupled to the AI-based computing system 104 via a network 106. In an embodiment of the present disclosure, the AI-based computing system 104 is a robotic system. Further, the set of data capturing devices 102 include one or more image capturing devices, one or more audio capturing devices, one or more sensors or a combination thereof. For example, the one or more audio capturing devices may be a microphone. The one or more image capturing devices may be a digital camera, smartphone, and the like. In an exemplary embodiment of the present disclosure, the one or more sensors may be a Light Detection and Ranging (LiDAR) scanner, microwave sensors, vibration motion sensors, ultrasonic motion sensors and the like. The network 106 may be an internet connection or any other wired or wireless network.

Further, the computing environment 100 includes one or more user devices 108 associated with the one or more users communicatively coupled to the AI-based computing system 104 via the network 106. The one or more user devices 108 are used by the one or more users to receive one or more notifications corresponding to falls detected, medical history, medication reminders, charging status and location of the AI-based computing system 104, vital scans performed, upcoming doctor appointments and the like. In an exemplary embodiment of the present disclosure, the one or more user devices 108 may include a laptop computer, desktop computer, tablet computer, smartphone, wearable device, smart watch, a digital camera and the like.

Furthermore, the computing environment 100 includes one or more electronic devices 110 associated with emergency contacts of the one or more users communicatively coupled to the AI-based computing system 104 via the network 106. In an exemplary embodiment of the present disclosure, the energy contacts include a health care professional, parent, spouse, child, friend of the one or more users and the like. The one or more electronic devices 110 are used by the emergency contacts to receive one or more alerts associated with the one or more users. In an embodiment of the present disclosure, the one or more alerts are received via text messages, voice calls and the like. For example, the one or more alerts corresponds to a dangerous accident associated with the one or more users, such as falling. In an exemplary embodiment of the present disclosure, the one or more electronic devices 110 may include a laptop computer, desktop computer, tablet computer, smartphone, wearable device, smart watch, a digital camera and the like.

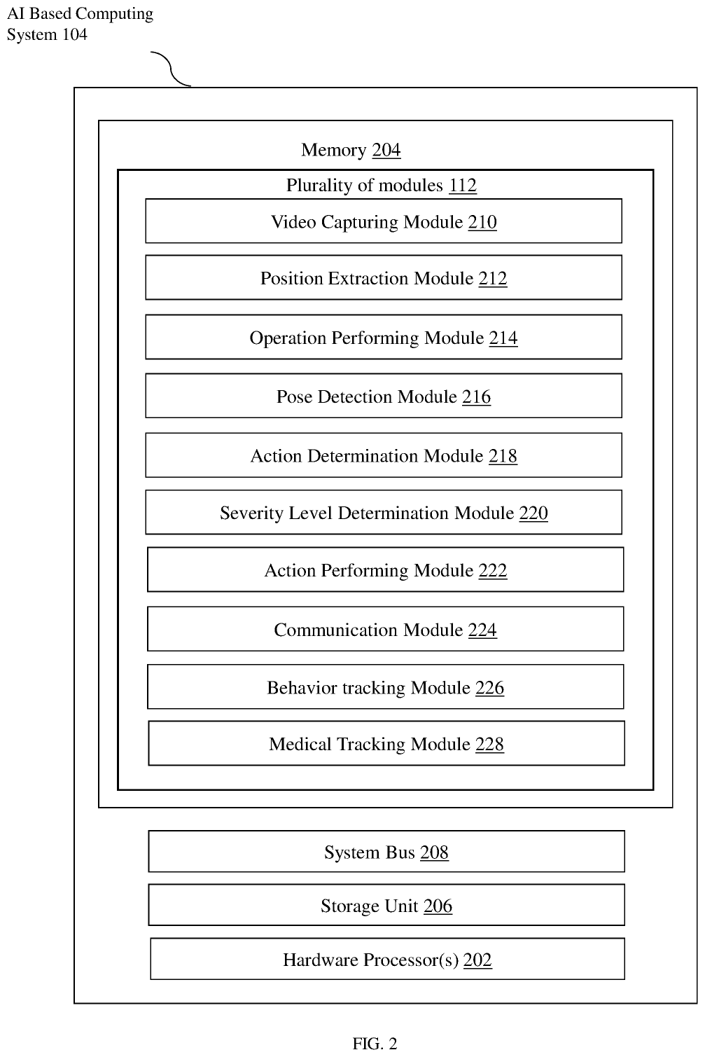

Further, the one or more user devices 108 include a local browser, a mobile application or a combination thereof. Furthermore, the one or more users may use a web application via the local browser, the mobile application or a combination thereof to communicate with the AI-based computing system 104 and receive the one or more notifications. In an exemplary embodiment of the present disclosure, the mobile application may be compatible with any mobile operating system, such as android, iOS, and the like. In an embodiment of the present disclosure, the AI-based computing system 104 includes a plurality of modules 112. Details on the plurality of modules 112 have been elaborated in subsequent paragraphs of the present description with reference to FIG. 2.

In an embodiment of the present disclosure, the AI-based computing system 104 is configured to capture one or more videos of the one or more users by using the one or more image capturing devices. Further, a plurality of frames are extracted from each of the captured one or more videos by using a frame extraction technique. Further, the AI-based computing system 104 extracts a set of skeletal positions from each of the extracted plurality of frames by using a position extraction-based AI model. The AI-based computing system 104 performs one or more operations on the extracted plurality of frames based on the extracted set of skeletal positions and one or more image parameters to normalize the extracted set of skeletal positions. Furthermore, the AI-based computing system 104 detects a set of poses of the one or more users in the plurality of frames based on the normalized set of skeletal positions and predefined pose information by using an action determination-based AI model. The AI-based computing system 104 determines an action from one or more actions performed by the one or more users in the extracted plurality of frames based on the normalized set of skeletal positions, the detected set of poses and the predefined pose information by using the action determination-based AI model. The AI-based computing system 104 determines a level of severity of the determined action based on reaction of the one or more users by using an offline voice management-based AI model. Further, the AI-based computing system 104 performs one or more responsive actions if the determined level of severity is above a predefined threshold level based on the determined action and the determined level of severity to provide medical assistance to the one or more users.

FIG. 2 is a block diagram illustrating an exemplary AI-based computing system 104 for automatically monitoring the health of one or more users, in accordance with an embodiment of the present disclosure. Further, the AI-based computing system 104 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 112 in the form of programmable instructions executable by the one or more hardware processors 202. Further, the plurality of modules 112 includes a video capturing module 210, a position extraction module 212, an operation performing module 214, a pose detection module 216, an action determination module 218, severity level determination module 220, an action performing module 222, a communication module 224, a behavior tracking module 226 and a medical tracking module 228.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 112 stored in the form of machine-readable instructions on any of the above-mentioned storage media and may be in communication with and executed by the one or more hardware processors 202.

In an embodiment of the present disclosure, the storage unit 206 may be a microchip, such that fall detection, speech recognition, conversational abilities and the like are specifically engineered to achieve edge-computing and working on-chip. Thus, the AI-based computing system 104 alleviates security, privacy and data concerns of the one or more users. Further, AI-based models, such as a position extraction-based AI model, an action determination based AI model, a rolling prediction based AI model, an offline voice management based AI model, an activity tracking-based AI model and the like are trained and deployed on the microchip of the AI-based computing system 104. The microchip may be a processor chip. In another embodiment of the present disclosure, the storage unit 206 may be a cloud storage. The AI-based computing system 104 may take consent of the one or more users before storing any content in the cloud storage. The storage unit 206 may store the one or more videos of the one or more users, the extracted set of skeletal positions, the normalized set of skeletal positions, the determined action, the level of severity, health data associated with the one or more users, one or more anomalies, the predefined pose information, the predefine threshold level, predefined medical information and the like.

The video capturing module 210 is configured to capture the one or more videos of the one or more users by using one or more image capturing devices. In an embodiment of the present disclosure, the one or more image capturing devices are placed inside the AI-based computing system 104. When the one or more image capturing devices are placed inside the AI-based computing system 104, the one or more image capturing devices capture the one or more videos in vicinity of the AI-based computing system 104. In another embodiment of the present disclosure, the one or more image capturing devices are placed in proximity of the one or more users. For example, the one or more image capturing devices are fixed on interior house walls of the one or more users to capture the one or more videos. The one or more users may include any class of people, such as elderly people, teenagers, disabled people and the like. In an embodiment of the present disclosure, the AI-based computing system 104 corresponds to a robotic system. For example, the robotic system may be a humanoid robot. In an exemplary embodiment of the present disclosure, the one or more image capturing devices may be digital camera, smartphone, and the like. Further, the plurality of frames are extracted from each of the captured one or more videos by using a frame extraction technique. The frame extraction technique involves reading the captured one or more videos from the one or more image capturing devices onboarded on the robotic system with determined frames per second parameter. In an embodiment of the present disclosure, the captured one or more videos are processed a frame at a time.

The position extraction module 212 is configured to extract the set of skeletal positions from each of the extracted plurality of frames by using the position extraction-based Artificial Intelligence (AI) model. The set of skeletal positions are subject to change as parameters are adjusted. For example, the set of skeletal positions may be any or all joints of human skeleton. In an exemplary embodiment of the present disclosure, the position extraction-based AI model is a convolution neural network model.

The operation performing module 214 is configured to perform the one or more operations on the extracted plurality of frames based on the extracted set of skeletal positions and one or more image parameters to normalize the extracted set of skeletal positions. The one or more operations include creating a bounding box around the one or more users in the extracted plurality of frames based on the extracted set of skeletal positions and the one or more image parameters. In an exemplary embodiment of the present disclosure, the one or more image parameters include distance of the one or more users from the one or more image capturing devices in the extracted plurality of frames, number of pixels in each of the extracted plurality of frames, distortions in the extracted plurality of frames, one or more camera angles in the extracted plurality of frames and the like. Further, the one or more operations include scaling a set of new data points in the created bounding box based on the extracted set of skeletal positions and the one or more image parameters. In an embodiment of the present disclosure, the set of new data points are skeletal points from extracted pose transformed to be relative to cropped or bounding box view. The one or more operations also include cropping the extracted plurality of frames upon scaling the set of new data points to normalize the extracted set of skeletal positions. In an embodiment of the present disclosure, when a user is near or far from the one or more image capturing devices, the user proportionally takes up more or fewer pixels respectively. Thus, coordinates of the user's pose are affected by distance to the one or more image capturing devices. Further, when the one or more operations are performed on the extracted plurality of frames, irrespective of the user's distance from the one or more image capturing devices, the user represents the same proportion. Thus, the one or more operations reduce noise and facilitate determination of action.

The pose detection module 216 is configured to detect the set of poses of the one or more users in the plurality of frames based on the normalized set of skeletal positions and predefined pose information by using the action determination-based AI model. In an embodiment of the present disclosure, the detected set of poses are added to a pose buffer.

The action determination module 218 is configured to determine an action from the one or more actions performed by the one or more users in the extracted plurality of frames based on the normalized set of skeletal positions, the detected set of poses and the predefined pose information by using the action determination based AI model. In an exemplary embodiment of the present disclosure, the one or more actions include sitting, standing, slipping, tripping, falling and the like. For example, the action is slipping of a user and hitting his head with a wall. In an embodiment of the present disclosure, the action determination-based AI model is a custom pre-trained neural network model. For example, the action determination-based AI model is trained on datasets, such as Common Objects In Context (COCO), Max-Planck-Institut für Informatik Human Pose Dataset (MPII) and the like. In an embodiment of the present disclosure, the action determination-based AI model is trained on proprietary data with custom data processing and cleaning to achieve uniqueness and accuracy. In determining the action from the one or more actions performed by the one or more users in the extracted plurality of frames based on the normalized set of skeletal positions, the detected set of poses and the predefined pose information by using the action determination based AI model, the action determination module 218 correlates the normalized set of skeletal positions, the detected set of poses of the one or more users in the extracted plurality of frames and the predefined pose information by using the action determination based AI model. In an embodiment of the present disclosure, the action determination-based AI model executes in the robotic system and does not require any internet or external connections to address privacy concerns. Further, the action determination module 218 generates a percentage value for each of the one or more one or more actions in the extracted plurality of frames based on result of correlation. The action determination module 218 determines the action from the one or more actions performed by the one or more users in the extracted plurality of frames based on the generated percentage value. The determined action has maximum percentage value. In an embodiment of the present disclosure, a set of frames from the plurality of frames are considered while determining the action. The set of frames are last frames of the plurality of frames stored in the pose buffer where the actual action takes place. For example, the set of frames are last n number of frames representing a person sitting, standing, falling or the like. In an embodiment of the present disclosure, the last n number of frames are used to determine the action across a short period instead of a single frame. The determined action is stored in an advanced buffer to determine accurate action using a rolling prediction-based AI model.

In an embodiment of the present disclosure, the action determination module 218 is configured to calculate average value of one or more percentage values associated with the one or more actions for the set of frames from the plurality of frames by using the rolling prediction-based AI model. The action determination module 218 determines the action from the one or more actions performed by the one or more users based on the calculated average value by using the rolling prediction-based AI model. For example, the rolling prediction-based AI model is is a Long Short-Term Memory Recurrent Neural Network (RNN) model. In an embodiment of the present disclosure, calculations, such as rolling average is performed on the advanced buffer to smooth any analogous readings for determining accurate action.

The severity level determination module 220 is configured to determine the level of severity of the determined action based on reaction of the one or more users by using the offline voice management-based AI model. In determining the level of severity of the determined action based on reaction of the one or more users, the severity level determination module 220 determines if the determined action corresponds to one or more hazardous actions. In an exemplary embodiment of the present disclosure, the one or more hazardous actions include slipping, tripping and falling. Further, the severity level determination module 220 determines if one or more audio responses are received from the one or more users upon determining that the determined action corresponds to the one or more hazardous actions. In an embodiment of the present disclosure, the one or more audio responses are received from the one or more audio capturing devices. For example, the one or more audio capturing devices may be microphone. In an embodiment of the present disclosure, the one or more audio capturing devices are placed inside the AI-based computing system 104 or placed in proximity of the one or more users. The severity level determination module 220 transcribes the one or more audio responses into one or more text responses by using the offline voice management-based AI model upon determining that the one or more audio responses are received from the one or more users. Furthermore, the severity level determination module 220 determines meaning and emotion of the received one or more audio inputs by applying sentiment analysis technique on the one or more text responses by using the offline voice management-based AI model. The severity level determination module 220 detects one or more emergency triggers in the one or more text responses by using the offline voice management-based AI model. For example, the one or more emergency triggers include help, call ambulance and the like. Further, the severity level determination module 220 determines the level of severity of the determined action based on the determined meaning, the determined emotion and the detected one or more emergency triggers by using the offline voice management-based AI model. In an exemplary embodiment of the present disclosure, the level of severity include grave, extremely critical, critical, serious and not hurt. In an embodiment of the present disclosure, the level of severity of the determined action is grave if the one or more audio responses are not received from the one or more users. The determined action and the determined level of severity may be outputted on the one or more user devices 108 associated with the one or more users. In an exemplary embodiment of the present disclosure, the one or more user devices 108 may include a laptop computer, desktop computer, tablet computer, smartphone, wearable device, smart watch, a digital camera and the like.

The action performing module 222 is configured to perform one or more responsive actions if the determined level of severity is above a predefined threshold level based on the determined action and the determined level of severity to provide medical assistance to the one or more users. The one or more responsive actions include outputting one or more alerts associated with the determined action and the severity level on the one or more electronic devices 110 associated with emergency contacts of the one or more users. In an exemplary embodiment of the present disclosure, the emergency contacts include health care professional, parent, spouse, child, friend of the one or more users and the like. In an exemplary embodiment of the present disclosure, the one or more electronic devices 110 may include a laptop computer, desktop computer, tablet computer, smartphone, wearable device, smart watch, a digital camera and the like. Further, the one or more responsive actions include calling emergency services to receive medical assistance for the one or more users. The one or more responsive actions also include determining position of the one or more users by using one or more sensors and Simultaneous Localization and Mapping (SLAM) to provide medical aid to the one or more users. When the position of the one or more users may be determined, the AI-based computing system 104 may reach the determined position, such that the one or more users may use ergonomically placed grips of the AI-based computing system 104 to seek assistance, such to stand-up in case of fall. In an exemplary embodiment of the present disclosure, the one or more sensors include Light Detection and Ranging (LiDAR) scanner, microwave sensors, vibration motion sensors, ultrasonic motion sensors and the like. In an embodiment of the present disclosure, the one or more sensors are placed inside the AI-based computing system 104 or placed in proximity of the one or more users.

In an embodiment of the present disclosure, the action performing module 222 receives one or more audio inputs from the one or more users by using the one or more audio capturing devices. The action performing module 222 detects if internet connection is available. Further, the action performing module 222 converts the received one or more audio inputs into one or more text outputs by using the offline voice management-based AI model upon detecting that the internet connection is not available. The action performing module 222 also determines meaning and emotion of the received one or more audio inputs by applying sentiment analysis technique on the one or more text outputs by using the offline voice management-based AI model. In an embodiment of the present disclosure, the sentiment analysis technique corresponds to natural language processing and text processing techniques to determine emotion of speech. Furthermore, the action performing module 222 detects one or more emergency triggers in the one or more text outputs by using the offline voice management-based AI model. The action performing module 222 performs the one or more responsive actions based on the determined action, determined emotion and the detected one or more emergency triggers by using the offline voice management-based AI model. For example, offline voice management-based AI model is a natural language processing model. In an embodiment of the present disclosure, the offline voice management-based AI model is a simplified model running on the AI-based computing system 104 for facilitating performance of the one or more responsive actions in case of simple request or emergencies. Thus, the AI-based computing system 104 act as redundancy system in event of losing the internet connection to still able to invoke emergency services or call for help from spoken requests. For example, the action performing module 222 detects the one or more emergency triggers, such as help, call ambulance, call Joe and the like, to take intermediate action, such as calling ambulance. In an embodiment of the present disclosure, the action performing module 222 also converts text to speech. In an exemplary embodiment of the present disclosure, python libraries and customized model, such as pyttsx3 may be used to convert text to speech.

The communication module 224 is configured to convert the received one or more audio inputs into the one or more text outputs by using a cloud voice management-based AI model upon detecting that the internet connection is available. In an exemplary embodiment of the present disclosure, the one or more audio inputs are transcribed into the one or more text outputs by using Amazon Transcribe, IBM Watson Speech to Text and the like. Further, the communication module 224 determines meaning and emotion of the received one or more audio inputs by applying sentiment analysis technique on the one or more text outputs by using the cloud voice management-based AI model. In an exemplary embodiment of the present disclosure, the cloud voice management-based AI model is a natural language processing model. The communication module 224 determines one or more best responses of the received one or more audio inputs based on the determined meaning and the determined emotion by using the cloud voice management-based AI model. In an embodiment of the present disclosure, OpenAI GP3 model is used for determining the one or more best responses. Furthermore, the communication module 224 converts the determined one or more best responses into one or more speech outputs by using the cloud voice management-based AI model. In an embodiment of the present disclosure, the one more best responses are converted into the one or more speech outputs by using services, such as Amazon Polly, IBM Watson Text to Speech and the like. The communication module 224 outputs the converted one or more speech outputs to the one or more users. In an embodiment of the present disclosure, the AI-based computing system 104 engage in a conversation to continue topics brought up or provide support if the detected emotion is upset, distress or the like. Thus, the communication module 224 continuously determines responses of audio inputs and converts the determined responses into speech outputs to maintain conversation with the one or more users. In an embodiment of the present disclosure, the cloud voice management-based AI model enables the AI-based computing system 104 to understand and respond to complex human speech pattern for providing emotional support and acting as a smart assistant, when connected to the internet.

In an embodiment of the present disclosure the AI-based computing system 104 is equipped with a wireless charging docking station, such that the one or more users are not required to worry about battery level of the AI-based computing system 104 and corresponding functionality. The AI-based computing system 104 may identify low battery level and may reach the wireless charging docking station automatically. In an embodiment of the present disclosure, the AI-based computing system 104 is equipped with internet-powered voice bot, such as Alexa, Google, IMB and the like for expansion of voice capabilities. The AI-based computing system 104 may also perform vital scans on the one or more users, such as blood pressure, SpO2 levels, blood glucose and the like. In an embodiment of the present disclosure, result of the vital scans are outputted on the one or more user devices 108. The one or more users may also confirm if the results of the vitals scans are in normal range using the AI-based computing system 104. The one or more users may also receive notifications corresponding to speed of Wireless Fidelity (Wi-Fi), battery of the AI-based computing system 104, speed of the AI-based computing system 104, information associated with the emergency contacts, number of devices connected via Bluetooth and the like on the one or more user devices 108. In an exemplary embodiment of the present disclosure, the information associated with the emergency contacts include name of emergency contact, relation of the emergency contact with the one or mor users, contact number of the emergency contact and the like. In an embodiment of the present disclosure, the one or more users may also receive notifications associated with falls detected, upcoming doctor appointments, medication reminders, charging percentage of the AI-based computing system 104, location of the AI-based computing system 104 and the like on the one or more user devices 108. Further, each of the one or more users may have different levels of access, such as intermediate access, full access and the like. The one or more users may also view the medical history of the one or more users, medical records, vital records, live feed, device last used and the like by using the one or more user devices 108. In an embodiment of the present disclosure, the one or more users may add a new user by providing email address, contact number, age, name, medical condition of the new user and the like by using the one or more user devices 108.

The behavior tracking module 226 is configured to capture health data associated with the one or more users for a predefined period of time. In an embodiment of the present disclosure, the health data includes sleeping time, waking-up time, number of hours of sleep, time and dose of taking one or more medicines, exercise time, meals times, walking speed of the one or more users and the like. Further, the behavior tracking module 226 determines behavior pattern of the one or more users by monitoring the health data using an activity tracking-based AI model. In an embodiment of the present disclosure, computer vision technology and unsupervised learning is used for determining the behavior pattern. The behavior tracking module 226 generates one or more reminders corresponding to the health data associated with the one or more users based on the determined behavior pattern by using the activity tracking-based AI model. In an embodiment of the present disclosure, the one or more reminders are spoken reminders. For example, the one or more reminders include medicine reminders, sleep reminders, meal reminders and the like to ensure that the one or more users are performing all tasks in a timely manner The behavior tracking module 226 captures one or more user activities associated with the one or more users. In an embodiment of the present disclosure, the one or more user activities are captured by using the set of data capturing devices 102. In an exemplary embodiment of the present disclosure, the set of data capturing devices 102 include the one or more image capturing devices, one or more audio capturing devices, one or more sensors or any combination thereof. For example, the one or more user activities include number of sleeping hours, time at which a user woke, number of hours slept, number of meals consumed by the user and the like. Furthermore, the behavior tracking module 226 determines one or more anomalies in the determined behavior pattern of the one or more users by comparing the captured one or more user activities with the determined behavior pattern by using the activity tracking-based AI model. In an exemplary embodiment of the present disclosure, the one or more anomalies include sudden increase in motion of the one or more users, change in number of hours of sleep, change in sleeping time and waking-up time, skipping medicines, change in duration of exercise, skipping one or more meals and the like. The behavior tracking module 226 generates one or more medical alerts corresponding to the captured one or more user activities based on the determined one or more anomalies by using the activity tracking-based AI model. For example, the one or more medical alerts include please sleep at 9M, please take medicine thrice, don't skip meals and the like. The behavior tracking module 226 also output the one or more medical alerts to the one or more users, emergency contacts of the one or more users or a combination thereof. In an embodiment of the present disclosure, the one or medical alerts are outputted to the one or more users via the one or more user devices 108. Further, the one or more medical alerts are outputted to the emergency contacts via the one or more electronic devices 110.

The medical tracking module 228 is configured to receive one or more medical inputs from a health care professional, user authorized caregiver or a combination thereof corresponding to the one or more medicines of the one or more users. In an embodiment of the present disclosure, the one or more medical inputs include the one or more medicines, doses, time, side effects of the one or more medicines and the like. Further, the medical tracking module 228 determines if the one or more users are complying with the received one or more medical inputs by monitoring the one or more user activities by using the activity tracking-based AI model. The medical tracking module 228 generates one or more medical recommendations if the one or more users are not complying with the received one or more medical inputs by using the activity tracking-based AI model. For example, the one or more medical recommendations include please take medicine twice, don't skip the medicine and the like. In an embodiment of the present disclosure, the one or more medical recommendations facilitates the one or more users to comply with the one or more medical inputs. The medical tracking module 228 also determines one or more diseases suffered by the one or more users based on the one or more medical inputs and predefined medical information. Furthermore, the medical tracking module 228 performs one or more activities based on the determined one or more diseases and the predefined medical information by using the activity tracking-based AI model. In an exemplary embodiment of the present disclosure, the one or mor activities include playing music, displaying one or more videos for performing exercise to cure the one or more diseases, initiating conversation with the one or more users and the like. For example, when a determined disease suffered by the user is depression, the AI-based computing system 104 plays music to calm down the user.

In an embodiment of the present disclosure, the AI-based computing system 104 includes the ergonomically placed grips with weight balancing techniques to withstand weight of the one or more users while seeking assistance. Further, the AI-based computing system 104 also includes a set of motors for locomotion of the AI-based computing system 104. The one or more image capturing devices are used for determining the action of the one or more users in the captured one or more videos. In an embodiment of the present disclosure, the one or more sensors are located between base and middle part of the AI-based computing system 104 for SLAM. Furthermore, the one or more audio capturing devices facilitates in conversational capabilities.

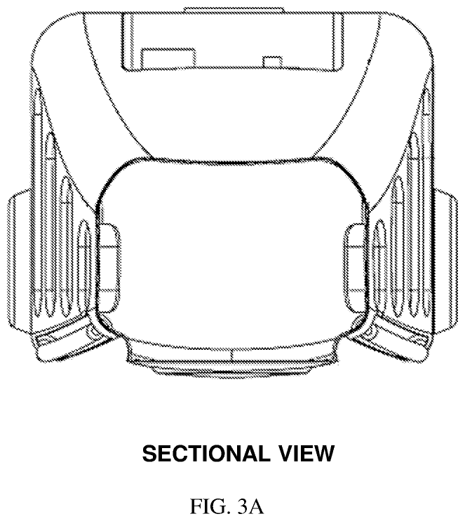

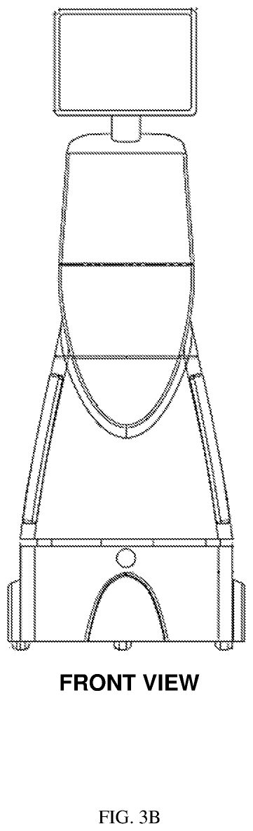

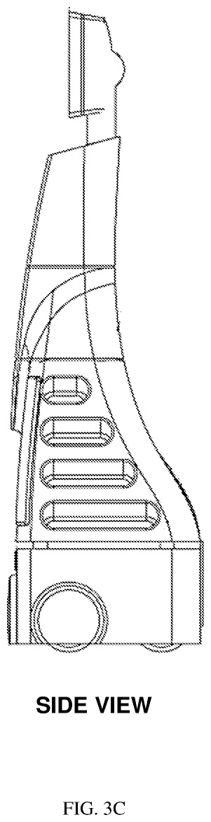

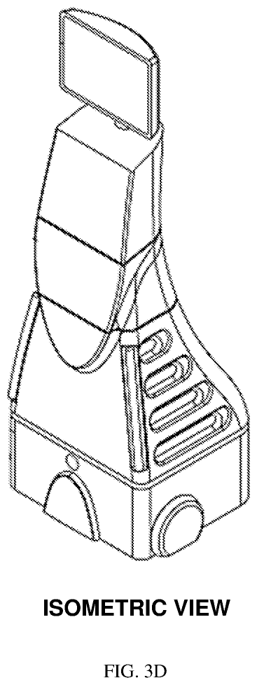

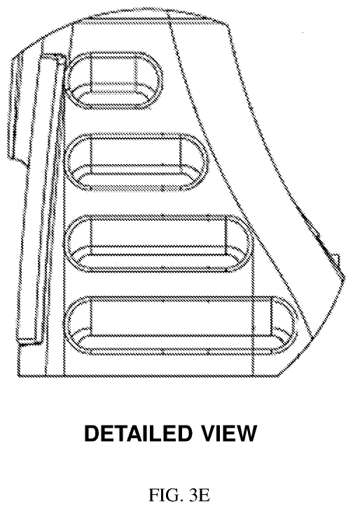

FIG. 3A is a top or sectional view of the exemplary AI-based computing system 104, in accordance with an embodiment of the present disclosure. Further, FIG. 3B is a front view of the exemplary AI-based computing system 104, in accordance with an embodiment of the present disclosure. FIG. 3C is a side view of the exemplary AI-based computing system 104, in accordance with an embodiment of the present disclosure. Furthermore, FIG. 3D is an isometric view of the exemplary AI-based computing system 104, in accordance with an embodiment of the present disclosure. FIG. 3E is a detailed view of exemplary ergonomically placed grips, in accordance with an embodiment of the present disclosure;

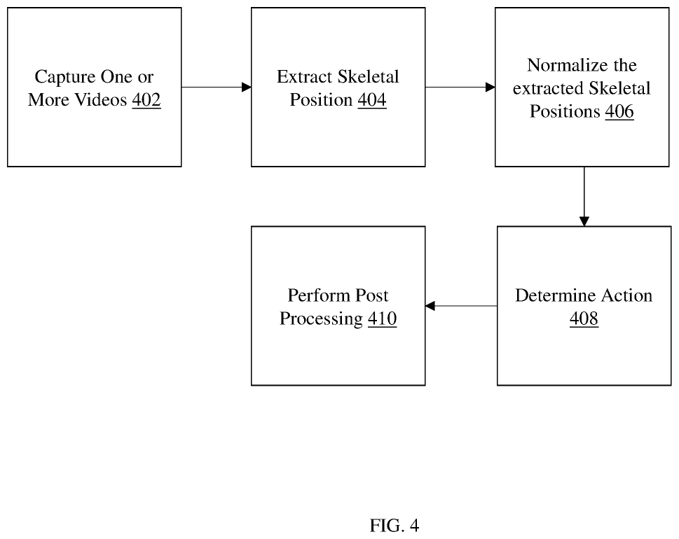

FIG. 4 is an exemplary block diagram depicting operation of the AI-based computing system 104 to determine the action from the one or more actions, in accordance with an embodiment of the present disclosure. In block 402, the AI-based computing system 104 captures the one or more videos of the one or more users by using the one or more image capturing devices. Further, the plurality of frames are extracted from each of the captured one or more videos by using the frame extraction technique. In block 404, the AI-based computing system 104 extracts the set of skeletal positions from each of the extracted plurality of frames by using the position extraction-based Artificial Intelligence (AI) model. In block 406, the AI-based computing system 104 performs one or more operations on the extracted plurality of frames based on the extracted set of skeletal positions and the one or more image parameters to normalize the extracted set of skeletal positions. Furthermore, the set of poses of the one or more users in the plurality of frames are detected based on the normalized set of skeletal positions and the predefined pose information by using an action determination-based AI model. In block 408, the AI-based computing system 104 determines the action from the one or more actions performed by the one or more users in the extracted plurality of frames based on the normalized set of skeletal positions, the detected set of poses and the predefined pose information by using the action determination based AI model. In block 410, the AI-based computing system 104 performs post-processing by calculating the average value of the one or more percentage values associated with the one or more actions for the set of frames by using the rolling prediction based AI model. Further, the AI-based computing system 104 determines the action from the one or more actions performed by the one or more users based on the calculated average value by using the rolling prediction-based AI model.

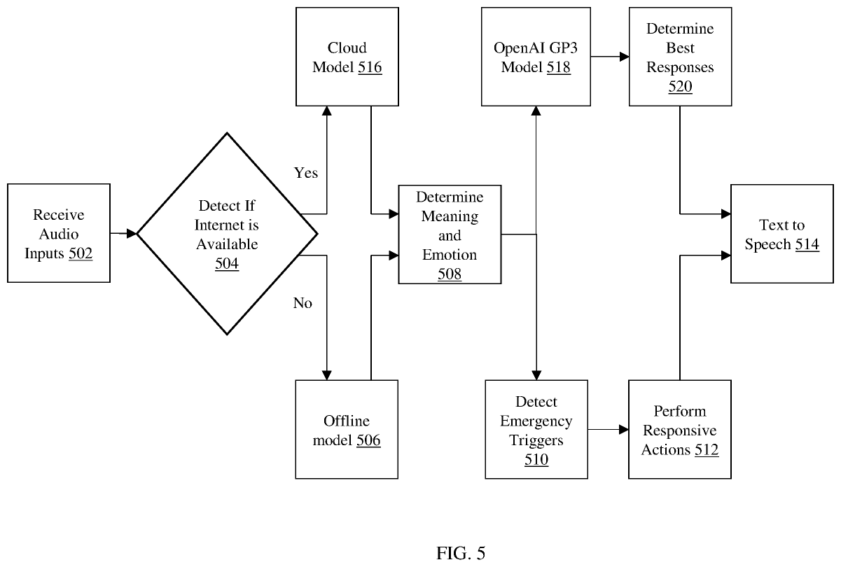

FIG. 5 is an exemplary block diagram depicting operation of the AI-based computing system 104 to perform the one or more responsive actions and output the one or more speech outputs, in accordance with an embodiment of the present disclosure. In block 502, the AI-based computing system 104 receives the one or more audio inputs from the one or more users by using the one or more audio capturing devices. Further, in block 504, the AI-based computing system 104 detects if the internet connection is available. In block 506, the AI-based computing system 104 converts the received one or more audio inputs into one or more text outputs by using the offline voice management-based AI model upon detecting that the internet connection is not available. In block 508, the AI-based computing system 104 determines meaning and emotion of the received one or more audio inputs by applying sentiment analysis technique on the one or more text outputs by using the offline voice management-based AI model. Furthermore, in block 510, the AI-based computing system 104 detects the one or more emergency triggers in the one or more text outputs by using the offline voice management-based AI model. In block 512, the AI-based computing system 104 performs the one or more responsive actions based on the determined action, determined emotion and the detected one or more emergency triggers by using the offline voice management-based AI model. In an embodiment of the present disclosure, in block 514, text is converted into speech.

Further, in block 516, the AI-based computing system 104 converts the received one or more audio inputs into the one or more text outputs by using the cloud voice management-based AI model upon detecting that the internet connection is available. In block 508, the AI-based computing system 104 determines meaning and emotion of the received one or more audio inputs by applying sentiment analysis technique on the one or more text outputs by using the cloud voice management-based AI model. In block 518, the AI-based computing system 104 uses an OpenAI GP3 model and in block 520, determines the one or more best responses of the received one or more audio inputs based on the determined meaning and the determined emotion by using the OpenAI GP3 model. Furthermore, in block 522, the AI-based computing system 104 converts the determined one or more best responses into the one or more speech outputs by using the cloud voice management-based AI model. In an embodiment of the present disclosure, the converted one or more speech outputs are outputted to the one or more users.

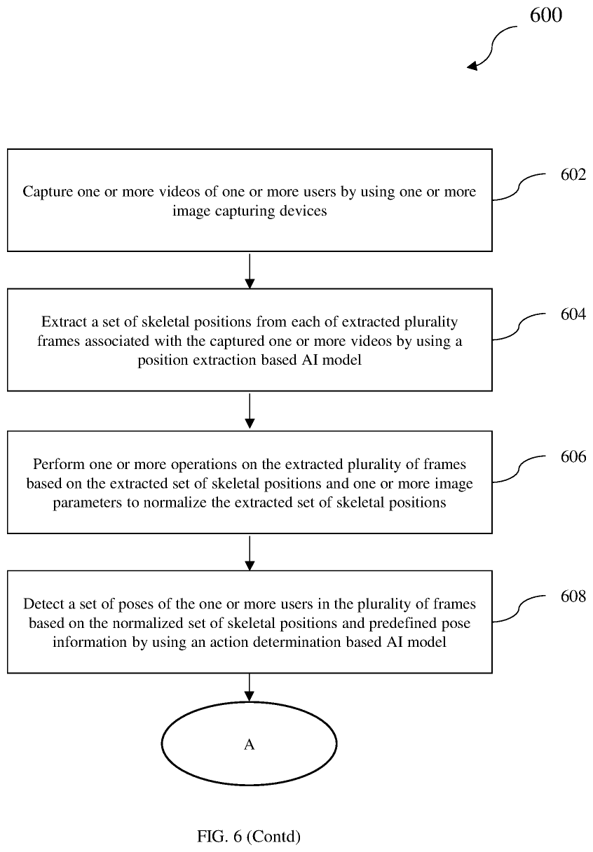

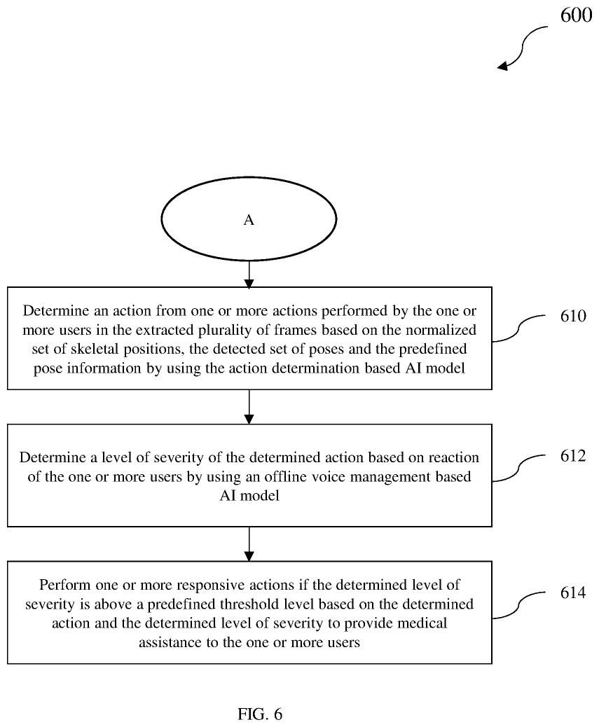

FIG. 6 is a process flow diagram illustrating an exemplary AI-based method for automatically monitoring the health of one or more users, in accordance with an embodiment of the present disclosure. At step 602, one or more videos of one or more users are captured by using one or more image capturing devices. In an embodiment of the present disclosure, the one or more image capturing devices are placed inside an AI-based computing system 104. When the one or more image capturing devices are placed inside the AI-based computing system 104, the one or more image capturing devices capture the one or more videos in vicinity of the AI-based computing system 104. In another embodiment of the present disclosure, the one or more image capturing devices are placed in proximity of the one or more users. For example, the one or more image capturing devices are fixed on interior house walls of the one or more users to capture the one or more videos. The one or more users may include any class of people, such as elderly people, teenagers, disabled people and the like. In an embodiment of the present disclosure, the AI-based computing system 104 corresponds to a robotic system. For example, the robotic system may be a humanoid robot. In an exemplary embodiment of the present disclosure, the one or more image capturing devices may be digital camera, smartphone, and the like. Further, a plurality of frames are extracted from each of the captured one or more videos by using a frame extraction technique. The frame extraction technique involves reading the captured one or more videos from the one or more image capturing devices onboarded on the robotic system with determined frames per second parameter. In an embodiment of the present disclosure, the captured one or more videos are processed a frame at a time. In an embodiment of the present disclosure, the AI-based method 600 is performed by the robotic system.

At step 604, a set of skeletal positions are extracted from each of the extracted plurality of frames by using a position extraction-based Artificial Intelligence (AI) model. In an exemplary embodiment of the present disclosure, the position extraction-based AI model is a convolution neural network model. The set of skeletal positions are subject to change as parameters are adjusted. For example, the set of skeletal positions may be any or all joints of human skeleton.

At step 606, one or more operations are performed on the extracted plurality of frames based on the extracted set of skeletal positions and one or more image parameters to normalize the extracted set of skeletal positions. The one or more operations include creating a bounding box around the one or more users in the extracted plurality of frames based on the extracted set of skeletal positions and the one or more image parameters. In an exemplary embodiment of the present disclosure, the one or more image parameters include distance of the one or more users from the one or more image capturing devices in the extracted plurality of frames, number of pixels in each of the extracted plurality of frames, distortions in the extracted plurality of frames, one or more camera angles in the extracted plurality of frames and the like. Further, the one or more operations include scaling a set of new data points in the created bounding box based on the extracted set of skeletal positions and the one or more image parameters. In an embodiment of the present disclosure, the set of new data points are skeletal points from extracted pose transformed to be relative to cropped or bounding box view. The one or more operations also include cropping the extracted plurality of frames upon scaling the set of new data points to normalize the extracted set of skeletal positions. In an embodiment of the present disclosure, when a user is near or far from the one or more image capturing devices, the user proportionally takes up more or fewer pixels respectively. Thus, coordinates of the user's pose are affected by distance to the one or more image capturing devices. Further, when the one or more operations are performed on the extracted plurality of frames, irrespective of the user's distance from the one or more image capturing devices, the user represents the same proportion. Thus, the one or more operations reduce noise and facilitate determination of action.

At step 608, a set of poses of the one or more users in the plurality of frames are detected based on the normalized set of skeletal positions and predefined pose information by using an action determination-based AI model. In an embodiment of the present disclosure, the detected set of poses are added to a pose buffer.

At step 610, an action from one or more actions performed by the one or more users in the extracted plurality of frames is determined based on the normalized set of skeletal positions, the detected set of poses and the predefined pose information by using the action determination based AI model. In an exemplary embodiment of the present disclosure, the one or more actions include sitting, standing, slipping, tripping, falling and the like. For example, the action is slipping of a user and hitting his head with a wall. In an embodiment of the present disclosure, the action determination-based AI model is a custom pre-trained neural network model. For example, the action determination-based AI model is trained on datasets, such as Common Objects In Context (COCO), Max-Planck-Institut für Informatik Human Pose Dataset (MPII) and the like. In an embodiment of the present disclosure, the action determination-based AI model is trained on proprietary data with custom data processing and cleaning to achieve uniqueness and accuracy. In determining the action from the one or more actions performed by the one or more users in the extracted plurality of frames based on the normalized set of skeletal positions, the detected set of poses and the predefined pose information by using the action determination based AI model, the AI-based method 600 includes correlating the normalized set of skeletal positions, the detected set of poses of the one or more users in the extracted plurality of frames and the predefined pose information by using the action determination based AI model. In an embodiment of the present disclosure, the action determination-based AI model executes in the robotic system and does not require any internet or external connections to address privacy concerns. Further, the AI-based method 600 includes generating a percentage value for each of the one or more one or more actions in the extracted plurality of frames based on result of correlation. The AI-based method 600 includes determining the action from the one or more actions performed by the one or more users in the extracted plurality of frames based on the generated percentage value. The determined action has maximum percentage value. In an embodiment of the present disclosure, a set of frames from the plurality of frames are considered while determining the action. The set of frames are last frames of the plurality of frames stored in the pose buffer where the actual action takes place. For example, the set of frames are last n number of frames representing a person sitting, standing, falling or the like. In an embodiment of the present disclosure, the last n number of frames are used to determine the action across a short period instead of a single frame. The determined action is stored in an advanced buffer to determine accurate action using a rolling prediction-based AI model.

In an embodiment of the present disclosure, the AI-based method 600 includes calculating average value of one or more percentage values associated with the one or more actions for the set of frames from the plurality of frames by using the rolling prediction-based AI model. The AI-based method 600 includes determining the action from the one or more actions performed by the one or more users based on the calculated average value by using the rolling prediction-based AI model. For example, the rolling prediction-based AI model is is a Long Short-Term Memory Recurrent Neural Network (RNN) model. In an embodiment of the present disclosure, calculations, such as rolling average is performed on the advanced buffer to smooth any analogous readings for determining accurate action.

At step 612, a level of severity of the determined action is determined based on reaction of the one or more users by using an offline voice management based AI model. In determining the level of severity of the determined action based on reaction of the one or more users, the AI-based method 600 includes determining if the determined action corresponds to one or more hazardous actions. In an exemplary embodiment of the present disclosure, the one or more hazardous actions include slipping, tripping and falling. Further, the AI-based method 600 includes determining if one or more audio responses are received from the one or more users upon determining that the determined action corresponds to the one or more hazardous actions. In an embodiment of the present disclosure, the one or more audio responses are received from the one or more audio capturing devices. For example, the one or more audio capturing devices may be microphone. In an embodiment of the present disclosure, the one or more audio capturing devices are placed inside the AI-based computing system 104 or placed in proximity of the one or more users. The AI-based method 600 includes transcribing the one or more audio responses into one or more text responses by using the offline voice management-based AI model upon determining that the one or more audio responses are received from the one or more users. Furthermore, the AI-based method 600 includes determining meaning and emotion of the received one or more audio inputs by applying sentiment analysis technique on the one or more text responses by using the offline voice management-based AI model. The AI-based method 600 includes detecting one or more emergency triggers in the one or more text responses by using the offline voice management-based AI model. For example, the one or more emergency triggers include help, call ambulance and the like. Further, the AI-based method 600 includes determining the level of severity of the determined action based on the determined meaning, the determined emotion and the detected one or more emergency triggers by using the offline voice management-based AI model. In an exemplary embodiment of the present disclosure, the level of severity include grave, extremely critical, critical, serious and not hurt. In an embodiment of the present disclosure, the level of severity of the determined action is grave if the one or more audio responses are not received from the one or more users. The determined action and the determined level of severity may be outputted on the one or more user devices 108 associated with the one or more users. In an exemplary embodiment of the present disclosure, the one or more user devices 108 may include a laptop computer, desktop computer, tablet computer, smartphone, wearable device, smart watch, a digital camera and the like.

At step 614, one or more responsive actions are performed if the determined level of severity is above a predefined threshold level based on the determined action and the determined level of severity to provide medical assistance to the one or more users. The one or more responsive actions include outputting one or more alerts associated with the determined action and the severity level on the one or more electronic devices 110 associated with emergency contacts of the one or more users. In an exemplary embodiment of the present disclosure, the emergency contacts include health care professional, parent, spouse, child, friend of the one or more users and the like. In an exemplary embodiment of the present disclosure, the one or more electronic devices 110 may include a laptop computer, desktop computer, tablet computer, smartphone, wearable device, smart watch, a digital camera and the like. Further, the one or more responsive actions include calling emergency services to receive medical assistance for the one or more users. The one or more responsive actions also include determining position of the one or more users by using one or more sensors and Simultaneous Localization and Mapping (SLAM) to provide medical aid to the one or more users. When the position of the one or more users may be determined, the AI-based computing system 104 may reach the determined position, such that the one or more users may use ergonomically placed grips of the AI-based computing system 104 to seek assistance, such to stand-up in case of fall. In an exemplary embodiment of the present disclosure, the one or more sensors include Light Detection and Ranging (LiDAR) scanner, microwave sensors, vibration motion sensors, ultrasonic motion sensors and the like. In an embodiment of the present disclosure, the one or more sensors are placed inside the AI-based computing system 104 or placed in proximity of the one or more users.

Further, the AI-based method 600 includes receiving one or more audio inputs from the one or more users by using the one or more audio capturing devices. The AI-based method 600 includes detecting if internet connection is available. Further, the AI-based method 600 includes converting the received one or more audio inputs into one or more text outputs by using the offline voice management-based AI model upon detecting that the internet connection is not available. The AI-based method 600 also includes determining meaning and emotion of the received one or more audio inputs by applying sentiment analysis technique on the one or more text outputs by using the offline voice management-based AI model. In an embodiment of the present disclosure, the sentiment analysis technique corresponds to natural language processing and text processing techniques to determine emotion of speech. Furthermore, the AI-based method 600 includes detecting one or more emergency triggers in the one or more text outputs by using the offline voice management-based AI model. The AI-based method 600 includes performing the one or more responsive actions based on the determined action, determined emotion and the detected one or more emergency triggers by using the offline voice management-based AI model. For example, offline voice management-based AI model is a natural language processing model. In an embodiment of the present disclosure, the offline voice management-based AI model is a simplified model running on the AI-based computing system 104 for facilitating performance of the one or more responsive actions in case of simple request or emergencies. For example, the AI-based method 600 includes detecting the one or more emergency triggers, such as help, call ambulance, call Joe and the like, to take intermediate action, such as calling ambulance. In an embodiment of the present disclosure, the AI-based method 600 also includes converting text to speech.

Furthermore, the AI-based method 600 includes converting the received one or more audio inputs into the one or more text outputs by using a cloud voice management-based AI model upon detecting that the internet connection is available. In an exemplary embodiment of the present disclosure, the one or more audio inputs are transcribed into the one or more text outputs by using Amazon Transcribe, IBM Watson Speech to Text and the like. Further, the AI-based method 600 includes determining meaning and emotion of the received one or more audio inputs by applying sentiment analysis technique on the one or more text outputs by using the cloud voice management-based AI model. In an exemplary embodiment of the present disclosure, the cloud voice management-based AI model is a natural language processing model. The AI-based method 600 includes determining one or more best responses of the received one or more audio inputs based on the determined meaning and the determined emotion by using the cloud voice management-based AI model. In an embodiment of the present disclosure, OpenAI GP3 model is used for determining the one or more best responses. Furthermore, the AI-based method 600 includes converting the determined one or more best responses into one or more speech outputs by using the cloud voice management-based AI model. In an embodiment of the present disclosure, the one more best responses are converted into the one or more speech outputs by using services, such as Amazon Polly, IBM Watson Text to Speech and the like. The AI-based method 600 includes outputting the converted one or more speech outputs to the one or more users. Thus, the AI-based method 600 includes continuously determining responses of audio inputs and converting the determined responses into speech outputs to maintain conversation with the one or more users. In an embodiment of the present disclosure, the cloud voice management-based AI model enables the functionality to understand and respond to complex human speech pattern for providing emotional support and acting as a smart assistant, when connected to the internet.

In an embodiment of the present disclosure, the AI-based method 600 includes identifying low battery level of the AI-based computing system 104 to reach a wireless charging docking station automatically. In an embodiment of the present disclosure, the AI-based computing system 104 is equipped with internet-powered voice bot, such as Alexa, Google, IMB and the like for expansion of voice capabilities. The AI-based method 600 includes performing vital scans on the one or more users, such as blood pressure, SpO2 levels, blood glucose and the like. In an embodiment of the present disclosure, result of the vital scans are outputted on the one or more user devices 108. The one or more users may also confirm if the results of the vitals scans are in normal range. The one or more users may also receive notifications corresponding to speed of Wireless Fidelity (Wi-Fi), battery of the AI-based computing system 104, speed of the AI-based computing system 104, information associated with the emergency contacts, number of devices connected via Bluetooth and the like on the one or more user devices 108. In an exemplary embodiment of the present disclosure, the information associated with the emergency contacts include name of emergency contact, relation of the emergency contact with the one or mor users, contact number of the emergency contact and the like. In an embodiment of the present disclosure, the one or more users may also receive notifications associated with falls detected, upcoming doctor appointments, medication reminders, charging percentage of the AI-based computing system 104, location of the AI-based computing system 104 and the like on the one or more user devices 108. Further, each of the one or more users may have different levels of access, such as intermediate access, full access and the like. The one or more users may also view the medical history of the one or more users, medical records, vital records, live feed, device last used and the like by using the one or more user devices 108. In an embodiment of the present disclosure, the one or more users may add a new user by providing email address, contact number, age, name, medical condition of the new user and the like by using the one or more user devices 108.

Furthermore, the AI-based method 600 includes capturing health data associated with the one or more users for a predefined period of time. In an embodiment of the present disclosure, the health data includes sleeping time, waking-up time, number of hours of sleep, time and dose of taking one or more medicines, exercise time, meals times, walking speed of the one or more users and the like. Further, the AI-based method 600 includes determining behavior pattern of the one or more users by monitoring the health data using an activity tracking-based AI model. In an embodiment of the present disclosure, computer vision technology and unsupervised learning is used for determining the behavior pattern. The AI-based method 600 includes generating one or more reminders corresponding to the health data associated with the one or more users based on the determined behavior pattern by using the activity tracking-based AI model. In an embodiment of the present disclosure, the one or more reminders are spoken reminders. For example, the one or more reminders include medicine reminders, sleep reminders, meal reminders and the like to ensure that the one or more users are performing all tasks in a timely manner The AI-based method 600 includes capturing one or more user activities associated with the one or more users. In an embodiment of the present disclosure, the one or more user activities are captured by using the set of data capturing devices 102. In an exemplary embodiment of the present disclosure, the set of data capturing devices 102 include the one or more image capturing devices, one or more audio capturing devices, one or more sensors or any combination thereof. For example, the one or more user activities include number of sleeping hours, time at which a user woke, number of hours slept, number of meals consumed by the user and the like. Furthermore, the AI-based method 600 includes determining one or more anomalies in the determined behavior pattern of the one or more users by comparing the captured one or more user activities with the determined behavior pattern by using the activity tracking-based AI model. In an exemplary embodiment of the present disclosure, the one or more anomalies include sudden increase in motion of the one or more users, change in number of hours of sleep, change in sleeping time and waking-up time, skipping medicines, change in duration of exercise, skipping one or more meals and the like. The AI-based method 600 includes generating one or more medical alerts corresponding to the captured one or more user activities based on the determined one or more anomalies by using the activity tracking-based AI model. For example, the one or more medical alerts include please sleep at 9M, please take medicine thrice, don't skip meals and the like. The AI-based method 600 also includes outputting the one or more medical alerts to the one or more users, emergency contacts of the one or more users or a combination thereof. In an embodiment of the present disclosure, the one or medical alerts are outputted to the one or more users via the one or more user devices 108. Further, the one or more medical alerts are outputted to the emergency contacts via the one or more electronic devices 110.

In an embodiment of the present disclosure, the AI-based method 600 includes receiving one or more medical inputs from a health care professional, user authorized caregiver or a combination thereof corresponding to the one or more medicines of the one or more users. In an embodiment of the present disclosure, the one or more medical inputs include the one or more medicines, doses, time, side effects of the one or more medicines and the like. Further, the AI-based method 600 includes determining if the one or more users are complying with the received one or more medical inputs by monitoring the one or more user activities by using the activity tracking-based AI model. The AI-based method 600 includes generating one or more medical recommendations if the one or more users are not complying with the received one or more medical inputs by using the activity tracking-based AI model. For example, the one or more medical recommendations include please take medicine twice, don't skip the medicine and the like. In an embodiment of the present disclosure, the one or more medical recommendations facilitates the one or more users to comply with the one or more medical inputs. The AI-based method 600 also includes determining one or more diseases suffered by the one or more users based on the one or more medical inputs and predefined medical information. Furthermore, the AI-based method 600 includes performing one or more activities based on the determined one or more diseases and the predefined medical information by using the activity tracking-based AI model. In an exemplary embodiment of the present disclosure, the one or mor activities include playing music, displaying one or more videos for performing exercise to cure the one or more diseases, initiating conversation with the one or more users and the like.

The AI-based method 600 may be implemented in any suitable hardware, software, firmware, or combination thereof.

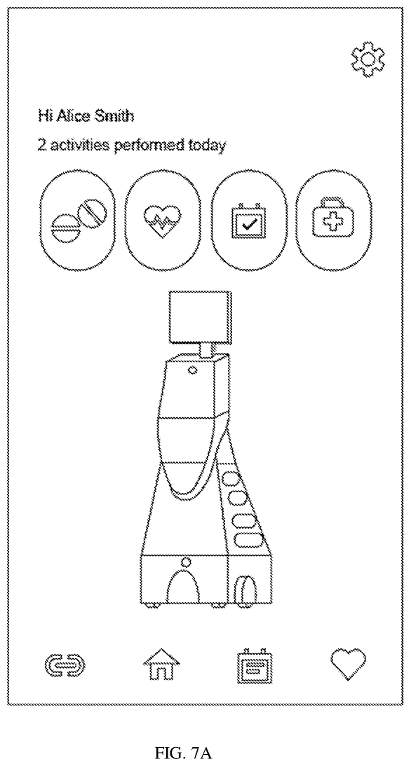

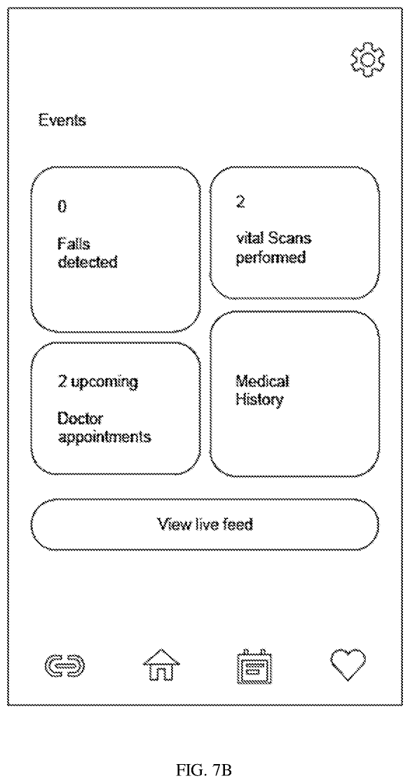

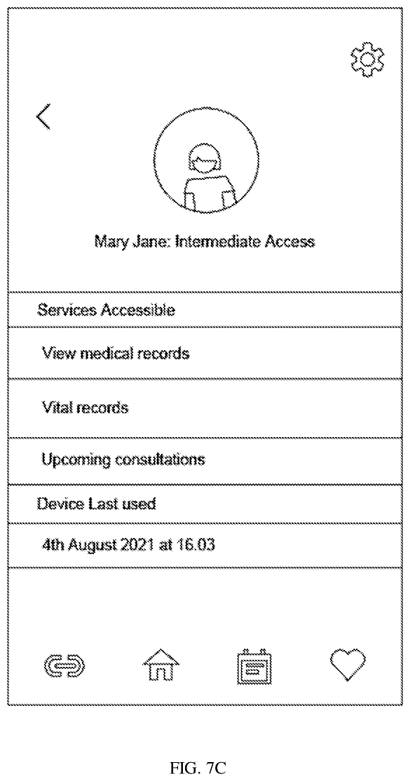

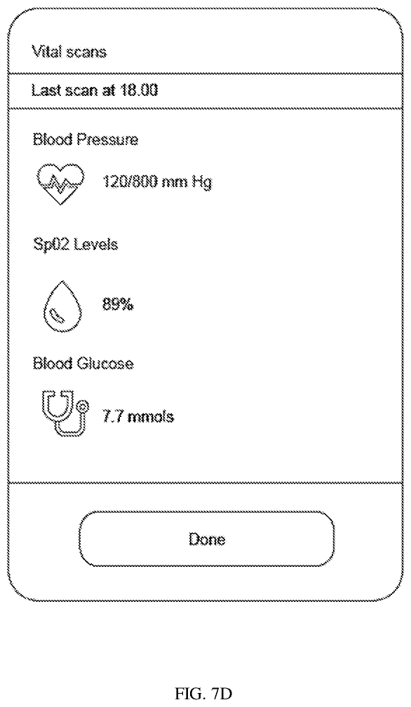

FIGS. 7A-7D are graphical user interface screens of the AI-based computing system 104 for automatically monitoring the health of the one or more users, in accordance with an embodiment of the present disclosure. FIG. 7A displays number of activities performed in a day, such as performing vital scans, medication tracking and the like. FIG. 7B displays events, such as number of falls detected, vital scans performed, number of upcoming doctor appointments and medical history of the one or more patients. The one or more users may also view live feed captured by the one or more image capturing devices. FIG. 7C displays a user profile including services available, medical records, vital records, upcoming consultations, devices last used and the like. FIG. 7D displays results of vital scans. For example, blood pressure of the user is 120/800 mm Hg, SpO2 levels of the users is 89% and blood glucose level of the user is 7.7 mmols.

Thus, various embodiments of the present AI-based computing system 104 provide a solution to automatically manage health of one or more users. The AI-based computing system 104 improves quality of life for elderly people by providing a-round-the-clock system that can flags anomalies, provide rapid attention to any fatal events, notify emergency contacts and essentially provide the best care for elderly. The AI-based computing system 104 is equipped with fall detection, speech recognition, conversational abilities and the like, such that the AI-based computing system 104 stands in the elderly or vulnerable environments as a first-line caregiver and a companion for the one or more users. In an embodiment of the present disclosure, the AI-based computing system 104 bridges gap between instant healthcare and the one or more users with a human-like product that not only makes sure they are taken care of but also converses with the one or more users emotionally to take care of mental health. Further, the AI-based computing system 104 also navigates to the one or more users in need. The AI-based computing system 104 detects falls with computer vision and navigates to fallen users to help them. Furthermore, the AI-based computing system 104 is capable of conversing with the one or more users and adhere to their needs as required. In an embodiment of the present disclosure, conversational abilities of the AI-based computing system 104 help to ease the one or more users with emotional health. The AI-based computing system 104 is equipped with indoor SLAM for seamless navigation to assist the user in need. In an embodiment of the present disclosure, fall detection, speech recognition, conversational abilities are specifically engineered to be edge-computing and working on-chip thereby alleviating the security, privacy and data concerns of the one or more users. Further, AI models are trained and deployed on the processor chip of the AI-based computing system 104 and left to the one or more users' discretion to store the AI models on the cloud storage. In an embodiment of the present disclosure, the robotic system may also be equipped with internet-powered voice bots for expansion of the voice capabilities. Furthermore, the AI-based computing system 104 may provide aid, call for emergency services in the event of a severe incident and the like. In an embodiment of the present disclosure, the action determination-based AI model is running on the AI-based computing system 104 and does not require any internet or external connections, to address privacy concerns. Furthermore, the cloud voice management-based AI model enables the AI-based computing system 104 to understand and respond to complex human speech pattern, provide emotional support and act as a smart assistant, when connected to the internet. Since loneliness is serious mental health issue for elders living alone, this feature aims to provide some form of support or engagement as the AI-based computing system 104 may discuss day to day matter and even hold debates. In an embodiment of the present disclosure, the AI-based computing system 104 may ensure that a user is remembering to take their medication using computer vision and spoken reminders. This ensures that the one or more users are not behind or over the prescribed amount. The AI-based computing system 104 is equipped with an unsupervised learning mechanism to draw patterns from the one or more users' daily routines and provide more insights to help serve the one or more users by personalizing the service. Furthermore, user management dashboard on mobile application or website portal of the AI-based computing system 104 may include tools to initialize and start monitoring the one or more users' medication. The user authorized caregiver or health care professional may enter the required medication, doses, time, side effects and the like. Further, the AI-based computing system 104 monitors the one or more users' activity, using computer vision and speech to track medication usage or engage the user to take medication. In an embodiment of the present disclosure, appropriate use of medication may be logged to ensure that the one or more users are not ahead or behind schedule.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 208 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

We claim:

1. An Artificial Intelligence (AI) based computing system for monitoring the health of one or more users, the AI-based computing system comprising:
one or more hardware processors; and
a memory coupled to the one or more hardware processors, wherein the memory comprises a plurality of modules in the form of programmable instructions executable by the one or more hardware processors, and wherein the plurality of modules comprises:
a video capturing module configured to capture one or more videos of one or more users by using one or more image capturing devices, wherein a plurality of frames are extracted from each of the captured one or more videos by using a frame extraction technique;
a position extraction module configured to extract a set of skeletal positions from each of the extracted plurality of frames by using a position extraction-based AI model;
an operation performing module configured to perform one or more operations on the extracted plurality of frames based on the extracted set of skeletal positions and one or more image parameters to normalize the extracted set of skeletal positions;
a pose detection module configured to detect a set of poses of the one or more users in the plurality of frames based on the normalized set of skeletal positions and predefined pose information by using an action determination-based AI model;
an action determination module configured to determine an action from one or more actions performed by the one or more users in the extracted plurality of frames based on the normalized set of skeletal positions, the detected set of poses, and the predefined pose information by using the action determination-based AI model, wherein the one or more actions comprise: sitting, standing, slipping, tripping, and falling;
a severity level determination module configured to determine a level of severity of the determined action based on reaction of the one or more users by using an offline voice management-based AI model; and
an action performing module configured to perform one or more responsive actions respondent to the determined level of severity being above a predefined threshold level based on the determined action and the determined level of severity to provide medical assistance to the one or more users.

2. The AI-based computing system of claim 1, wherein the AI-based computing system corresponds to a robotic system.

3. The AI-based computing system of claim 1, wherein the one or more operations comprises:
creating a bounding box around the one or more users in the extracted plurality of frames based on the extracted set of skeletal positions and the one or more image parameters, wherein the one or more image parameters comprises: distance of the one or more users from the one or more image capturing devices in the extracted plurality of frames, number of pixels in each of the extracted plurality of frames, distortions in the extracted plurality of frames, and one or more camera angles in the extracted plurality of frames;
scaling a set of new data points in the created bounding box based on the extracted set of skeletal positions and the one or more image parameters; and
cropping the extracted plurality of frames upon scaling the set of new data points to normalize the extracted set of skeletal positions.

4. The AI-based computing system of claim 1, wherein the one or more responsive actions comprise:
outputting one or more alerts associated with the determined action and the severity level on one or more electronic devices associated with emergency contacts of the one or more users, wherein the emergency contacts comprise: health care professional, parent, spouse, child, and friend of the one or more users;
calling emergency services to receive medical assistance for the one or more users; and
determining a position of the one or more users by using one or more sensors and Simultaneous Localization and Mapping (SLAM) to provide medical aid to the one or more users, wherein the one or more sensors comprise: Light Detection and Ranging (LiDAR) scanner, microwave sensors, vibration motion sensors, and ultrasonic motion sensors.

5. The AI-based computing system of claim 1, wherein in determining the action from the one or more actions performed by the one or more users in the extracted plurality of frames based on the normalized set of skeletal positions, the detected set of poses and the predefined pose information by using the action determination-based AI model, the action determination module is configured to:
correlate the normalized set of skeletal positions, the detected set of poses of the one or more users in the extracted plurality of frames, and the predefined pose information by using the action determination-based AI model;
generate a percentage value for each of the one or more actions in the extracted plurality of frames based on result of correlation; and
determine the action from the one or more actions performed by the one or more users in the extracted plurality of frames based on the generated percentage value, wherein the determined action has a maximum percentage value.

6. The AI-based computing system of claim 5, wherein the action determination module is configured to:
calculate an average value of one or more percentage values associated with the one or more actions for a set of frames from the plurality of frames by using a rolling prediction-based AI model; and
determine the action from the one or more actions performed by the one or more users based on the calculated average value by using the rolling prediction-based AI model.

7. The AI-based computing system of claim 1, wherein the action performing module is configured to:
receive one or more audio inputs from the one or more users by using one or more audio capturing devices;
detect whether an internet connection is available;
convert the received one or more audio inputs into one or more text outputs by using the offline voice management-based AI model upon detecting that the internet connection is not available;
determine meaning and emotion of the received one or more audio inputs by applying a sentiment analysis technique on the one or more text outputs by using the offline voice management-based AI model;
detect one or more emergency triggers in the one or more text outputs by using the offline voice management-based AI model; and
perform the one or more responsive actions based on the determined action, determined emotion and the detected one or more emergency triggers by using the offline voice management-based AI model.

8. The AI-based computing system of claim 7, further comprising a communication module configured to:
convert the received one or more audio inputs into the one or more text outputs by using a cloud voice management-based AI model upon detecting that the internet connection is available;
determine meaning and emotion of the received one or more audio inputs by applying the sentiment analysis technique on the one or more text outputs by using the cloud voice management-based AI model;
determine one or more best responses of the received one or more audio inputs based on the determined meaning and the determined emotion by using the cloud voice management-based AI model;
convert the determined one or more best responses into one or more speech outputs by using the cloud voice management-based AI model; and
output the converted one or more speech outputs to the one or more users.

9. The AI-based computing system of claim 1, further comprising a behavior tracking module configured to:
capture health data associated with the one or more users for a predefined period of time, wherein the health data comprises: sleeping time, waking-up time, number of hours of sleep, time and dose of taking one or more medicines, exercise time, meal times, and walking speed of the one or more users;
determine a behavior pattern of the one or more users by monitoring health data using an activity tracking-based AI model;
generate one or more reminders corresponding to health data associated with the one or more users based on the determined behavior pattern by using the activity tracking-based AI model;
capture one or more user activities associated with the one or more users, wherein the one or more user activities are captured by using a set of data capturing devices and wherein the set of data capturing devices comprise at least one of: the one or more image capturing devices, one or more audio capturing devices and one or more sensors;
determine one or more anomalies in the determined behavior pattern of the one or more users by comparing the captured one or more user activities with the determined behavior pattern by using the activity tracking-based AI model, wherein the one or more anomalies comprise:
sudden increase in motion of the one or more users, change in number of hours of sleep, change in sleeping time and waking-up time, skipping medicines, change in duration of exercise, and skipping one or more meals;
generate one or more medical alerts corresponding to the captured one or more user activities based on the determined one or more anomalies by using the activity tracking-based AI model; and
output the one or more medical alerts to at least one of: the one or more users and emergency contacts of the one or more users.

10. The AI-based computing system of claim 9, further comprising a medical tracking module configured to:
receive one or more medical inputs from at least one of: a health care professional and user authorized caregiver corresponding to the one or more medicines of the one or more users, wherein the one or more medical inputs comprise: the one or more medicines, doses, and time and side effects of the one or more medicines;
determine if the one or more users are complying with the received one or more medical inputs by monitoring the one or more user activities by using the activity tracking-based AI model;
generate one or more medical recommendations if the one or more users are not complying with the received one or more medical inputs by using the activity tracking-based AI model, wherein the one or more medical recommendations facilitates the one or more users to comply with the one or more medical inputs;
determine one or more diseases suffered by the one or more users based on the one or more medical inputs and predefined medical information; and
perform one or more activities based on the determined one or more diseases and the predefined medical information by using the activity tracking-based AI model, wherein the one or mor activities comprise: playing music, displaying one or more videos for performing exercise to cure the one or more diseases, and initiating conversation with the one or more users.

11. The AI-based computing system of claim 1, wherein in determining the level of severity of the determined action based on reaction of the one or more users, the severity level determination module is configured to:
determine if the determined action corresponds to one or more hazardous actions, wherein the one or more hazardous actions comprise slipping, tripping, and falling;
determine if one or more audio responses are received from the one or more users upon determining that the determined action corresponds to the one or more hazardous actions;
transcribe the one or more audio responses into one or more text responses by using the offline voice management-based AI model upon determining that the one or more audio responses are received from the one or more users;
determine meaning and emotion of the received one or more audio inputs by applying a sentiment analysis technique on the one or more text responses by using the offline voice management-based AI model;
detect one or more emergency triggers in the one or more text responses by using the offline voice management-based AI model; and
determine the level of severity of the determined action based on the determined meaning, the determined emotion and the detected one or more emergency triggers by using the offline voice management-based AI model, wherein the level of severity comprises: grave, extremely critical, critical, serious, and not hurt, and wherein the level of severity of the determined action is grave if the one or more audio responses are not received from the one or more users.

12. An Artificial Intelligence (AI) based method for automatically monitoring the health of one or more users, the AI-based method comprising:
capturing, by one or more hardware processors, one or more videos of one or more users by using one or more image capturing devices, wherein a plurality of frames are extracted from each of the captured one or more videos by using a frame extraction technique;
extracting, by the one or more hardware processors, a set of skeletal positions from each of the extracted plurality of frames by using a position extraction-based AI model;
performing, by the one or more hardware processors, one or more operations on the extracted plurality of frames based on the extracted set of skeletal positions and one or more image parameters to normalize the extracted set of skeletal positions;
detecting, by the one or more hardware processors, a set of poses of the one or more users in the plurality of frames based on the normalized set of skeletal positions and predefined pose information by using an action determination-based AI model;
determining, by the one or more hardware processors, an action from one or more actions performed by the one or more users in the extracted plurality of frames based on the normalized set of skeletal positions, the detected set of poses, and the predefined pose information by using the action determination-based AI model, wherein the one or more actions comprise: sitting, standing, slipping, tripping, and falling;
determining, by the one or more hardware processors, a level of severity of the determined action based on reaction of the one or more users by using an offline voice management-based AI model; and
performing, by the one or more hardware processors, one or more responsive actions if the determined level of severity is above a predefined threshold level based on the determined action and the determined level of severity to provide medical assistance to the one or more users.

13. The AI-based method of claim 12, wherein the AI-based method is performed by a robotic system.

14. The AI-based method of claim 12, wherein the one or more operations comprise:
creating a bounding box around the one or more users in the extracted plurality of frames based on the extracted set of skeletal positions and the one or more image parameters, wherein the one or more image parameters comprise: distance of the one or more users from the one or more image capturing devices in the extracted plurality of frames, number of pixels in each of the extracted plurality of frames, distortions in the extracted plurality of frames, and one or more camera angles in the extracted plurality of frames;
scaling a set of new data points in the created bounding box based on the extracted set of skeletal positions and the one or more image parameters; and
cropping the extracted plurality of frames upon scaling the set of new data points to normalize the extracted set of skeletal positions.

15. The AI-based method of claim 12, wherein the one or more responsive actions comprise:
outputting one or more alerts associated with the determined action and the severity level on one or more electronic devices associated with emergency contacts of the one or more users, wherein the emergency contacts comprise: health care professional, parent, spouse, child, and friend of the one or more users;
calling emergency services to receive medical assistance for the one or more users; and
determining position of the one or more users by using one or more sensors and Simultaneous Localization and Mapping (SLAM) to provide medical aid to the one or more users, wherein the one or more sensors comprise: Light Detection and Ranging (LiDAR) scanner, microwave sensors, vibration motion sensors, and ultrasonic motion sensors.

16. The AI-based method of claim 12, wherein determining the action from the one or more actions performed by the one or more users in the extracted plurality of frames based on the normalized set of skeletal positions, the detected set of poses and the predefined pose information by using the action determination-based AI model comprises:
correlating the normalized set of skeletal positions, the detected set of poses of the one or more users in the extracted plurality of frames and the predefined pose information by using the action determination-based AI model;
generating a percentage value for each of the one or more one or more actions in the extracted plurality of frames based on result of correlation; and
determining the action from the one or more actions performed by the one or more users in the extracted plurality of frames based on the generated percentage value, wherein the determined action has a maximum percentage value.

17. The AI-based method of claim 16, further comprising:
calculating an average value of one or more percentage values associated with the one or more actions for a set of frames from the plurality of frames by using a rolling prediction-based AI model; and
determining the action from the one or more actions performed by the one or more users based on the calculated average value by using the rolling prediction-based AI model.

18. The AI-based method of claim 12, further comprising:
receiving one or more audio inputs from the one or more users by using one or more audio capturing devices;
detecting if an internet connection is available;
converting the received one or more audio inputs into one or more text outputs by using an offline voice management-based AI model upon detecting that the internet connection is not available;
determining meaning and emotion of the received one or more audio inputs by applying a sentiment analysis technique on the one or more text outputs by using the offline voice management-based AI model;
detecting one or more emergency triggers in the one or more text outputs by using the offline voice management-based AI model; and
performing the one or more responsive actions based on the determined action, determined emotion, and the detected one or more emergency triggers by using the offline voice management-based AI model.

19. The AI-based method of claim 18, further comprising:
converting the received one or more audio inputs into the one or more text outputs by using a cloud voice management-based AI model upon detecting that the internet connection is available;
determining meaning and emotion of the received one or more audio inputs by applying the sentiment analysis technique on the one or more text outputs by using the cloud voice management-based AI model;
determining one or more best responses of the received one or more audio inputs based on the determined meaning and the determined emotion by using the cloud voice management-based AI model;
converting the determined one or more best responses into one or more speech outputs by using the cloud voice management-based AI model; and
outputting the converted one or more speech outputs to the one or more users.

20. The AI-based method of claim 12, further comprising:
capturing health data associated with the one or more users for a predefined period of time, wherein the health data comprises: sleeping time, waking-up time, number of hours of sleep, time and dose of taking one or more medicines, exercise time, meal times, and walking speed of the one or more users;
determining a behavior pattern of the one or more users by monitoring health data using an activity tracking-based AI model;
generating one or more reminders corresponding to the health data associated with the one or more users based on the determined behavior pattern by using the activity tracking-based AI model;
capturing one or more user activities associated with the one or more users, wherein the one or more user activities are captured by using a set of data capturing devices, and wherein the set of data capturing devices comprise at least one of: the one or more image capturing devices, one or more audio capturing devices, and one or more sensors;
determining one or more anomalies in the determined behavior pattern of the one or more users by comparing the captured one or more user activities with the determined behavior pattern by using the activity tracking-based AI model, wherein the one or more anomalies comprise:
sudden increase in motion of the one or more users, change in number of hours of sleep, change in sleeping time and waking-up time, skipping medicines, change in duration of exercise, and skipping one or more meals;
generating one or more medical alerts corresponding to the captured one or more user activities based on the determined one or more anomalies by using the activity tracking-based AI model; and
outputting the one or more medical alerts to at least one of: the one or more users and emergency contacts of the one or more users.

21. The AI-based method of claim 20, further comprising:
receiving one or more medical inputs from at least one of: a health care professional and user authorized caregiver corresponding to the one or more medicines of the one or more users, wherein the one or more medical inputs comprise: the one or more medicines, doses, and time and side effects of the one or more medicines;
determining if the one or more users are complying with the received one or more medical inputs by monitoring the one or more user activities by using the activity tracking-based AI model;
generating one or more medical recommendations if the one or more users are not complying with the received one or more medical inputs by using the activity tracking-based AI model, wherein the one or more medical recommendations facilitates the one or more users to comply with the one or more medical inputs;
determining one or more diseases suffered by the one or more users based on the one or more medical inputs and predefined medical information; and
performing one or more activities based on the determined one or more diseases and the predefined medical information by using the activity tracking-based AI model, wherein the one or mor activities comprise: playing music, displaying one or more videos for performing exercise to cure the one or more diseases, and initiating conversation with the one or more users.

22. The AI-based method of claim 12, wherein determining the level of severity of the determined action based on reaction of the one or more users comprises:
determining if the determined action corresponds to one or more hazardous actions, wherein the one or more hazardous actions comprise slipping, tripping, and falling;
determining if one or more audio responses are received from the one or more users upon determining that the determined action corresponds to the one or more hazardous actions;
transcribing the one or more audio responses into one or more text responses by using the offline voice management-based AI model upon determining that the one or more audio responses are received from the one or more users;
determining meaning and emotion of the received one or more audio inputs by applying a sentiment analysis technique on the one or more text responses by using the offline voice management-based AI model;
detecting one or more emergency triggers in the one or more text responses by using the offline voice management-based AI model; and
determining the level of severity of the determined action based on the determined meaning, the determined emotion, and the detected one or more emergency triggers by using the offline voice management-based AI model, wherein the level of severity comprises: grave, extremely critical, critical, serious, and not hurt, and wherein the level of severity of the determined action is grave if the one or more audio responses are not received from the one or more users.
